SEQUENCE LISTING (75 sequences)

SEQ ID NO: 1 - protein, 272 aa, synthetic construct
  REGION 1..272: note = N272 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGIN

SEQ ID NO: 2 - protein, 2583 aa, synthetic construct
  REGION 1..2583: note = C273 Fragment amino acid sequence
SERILELYSLEUPROLYSASPARGILEHISSERGLULYSSERASNLYSSERVALALAMETASPMETLEUASNGLUVALLYSARGCYSPROASPGLULEUPHETHRTHRLEUSERALAGLULYSGLNSERARGPHEARGILEILESERASPASPHISASNGLUVALLEUMETLYSARGSERSERASPARGPHEVALPROLEULEULEUGLNTYRILEASPTYRGLYLYSLEUPHEASPHISILEARGPHEHISVALASNMETGLYLYSLEUARGTYRLEULEULYSALAASPLYSTHRCYSILEASPGLYGLNTHRARGVALARGVALILEGLUGLNPROLEUASNGLYPHEGLYARGLEUGLUGLUALAGLUTHRMETARGLYSGLNGLUASNGLYTHRPHEGLYASNSERGLYILEARGILEARGASPPHEGLUASNMETLYSARGASPASPALAASNPROALAASNTYRPROTYRILEVALASPTHRTYRTHRHISTYRILELEUGLUASNASNLYSVALGLUMETPHEILEASNASPLYSGLUASPSERALAPROLEULEUPROVALILEGLUASPASPARGTYRVALVALLYSTHRILEPROSERCYSARGMETSERTHRLEUGLUILEPROALAMETALAPHEHISMETPHELEUPHEGLYSERLYSLYSTHRGLULYSLEUILEVALASPVALHISASNARGTYRLYSARGLEUPHEGLNALAMETGLNLYSGLUGLUVALTHRALAGLUASNILEALASERPHEGLYILEALAGLUSERASPLEUPROGLNLYSILELEUASPLEUILESERGLYASNALAHISGLYLYSASPVALASPALAPHEILEARGLEUTHRVALASPASPMETLEUTHRASPTHRGLUARGARGILELYSARGPHELYSASPASPARGLYSSERILEARGSERALAASPASNLYSMETGLYLYSARGGLYPHELYSGLNILESERTHRGLYLYSLEUALAASPPHELEUALALYSASPILEVALLEUPHEGLNPROSERVALASNASPGLYGLUASNLYSILETHRGLYLEUASNTYRARGILEMETGLNSERALAILEALAVALTYRASPSERGLYASPASPTYRGLUALALYSGLNGLNPHELYSLEUMETPHEGLULYSALAARGLEUILEGLYLYSGLYTHRTHRGLUPROHISPROPHELEUTYRLYSVALPHEALAARGSERILEPROALAASNALAVALGLUPHETYRGLUARGTYRLEUILEGLUARGLYSPHETYRLEUTHRGLYLEUSERASNGLUILELYSLYSGLYASNARGVALASPVALPROPHEILEARGARGASPGLNASNLYSTRPLYSTHRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 3 - protein, 286 aa, synthetic construct
  REGION 1..286: note = N286 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKS

SEQ ID NO: 4 - protein, 2541 aa, synthetic construct
  REGION 1..2541: note = C287 Fragment amino acid sequence
ASNLYSSERVALALAMETASPMETLEUASNGLUVALLYSARGCYSPROASPGLULEUPHETHRTHRLEUSERALAGLULYSGLNSERARGPHEARGILEILESERASPASPHISASNGLUVALLEUMETLYSARGSERSERASPARGPHEVALPROLEULEULEUGLNTYRILEASPTYRGLYLYSLEUPHEASPHISILEARGPHEHISVALASNMETGLYLYSLEUARGTYRLEULEULYSALAASPLYSTHRCYSILEASPGLYGLNTHRARGVALARGVALILEGLUGLNPROLEUASNGLYPHEGLYARGLEUGLUGLUALAGLUTHRMETARGLYSGLNGLUASNGLYTHRPHEGLYASNSERGLYILEARGILEARGASPPHEGLUASNMETLYSARGASPASPALAASNPROALAASNTYRPROTYRILEVALASPTHRTYRTHRHISTYRILELEUGLUASNASNLYSVALGLUMETPHEILEASNASPLYSGLUASPSERALAPROLEULEUPROVALILEGLUASPASPARGTYRVALVALLYSTHRILEPROSERCYSARGMETSERTHRLEUGLUILEPROALAMETALAPHEHISMETPHELEUPHEGLYSERLYSLYSTHRGLULYSLEUILEVALASPVALHISASNARGTYRLYSARGLEUPHEGLNALAMETGLNLYSGLUGLUVALTHRALAGLUASNILEALASERPHEGLYILEALAGLUSERASPLEUPROGLNLYSILELEUASPLEUILESERGLYASNALAHISGLYLYSASPVALASPALAPHEILEARGLEUTHRVALASPASPMETLEUTHRASPTHRGLUARGARGILELYSARGPHELYSASPASPARGLYSSERILEARGSERALAASPASNLYSMETGLYLYSARGGLYPHELYSGLNILESERTHRGLYLYSLEUALAASPPHELEUALALYSASPILEVALLEUPHEGLNPROSERVALASNASPGLYGLUASNLYSILETHRGLYLEUASNTYRARGILEMETGLNSERALAILEALAVALTYRASPSERGLYASPASPTYRGLUALALYSGLNGLNPHELYSLEUMETPHEGLULYSALAARGLEUILEGLYLYSGLYTHRTHRGLUPROHISPROPHELEUTYRLYSVALPHEALAARGSERILEPROALAASNALAVALGLUPHETYRGLUARGTYRLEUILEGLUARGLYSPHETYRLEUTHRGLYLEUSERASNGLUILELYSLYSGLYASNARGVALASPVALPROPHEILEARGARGASPGLNASNLYSTRPLYSTHRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 5 - protein, 351 aa, synthetic construct
  REGION 1..351: note = N351 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFD

SEQ ID NO: 6 - protein, 2346 aa, synthetic construct
  REGION 1..2346: note = C352 Fragment amino acid sequence
HISILEARGPHEHISVALASNMETGLYLYSLEUARGTYRLEULEULYSALAASPLYSTHRCYSILEASPGLYGLNTHRARGVALARGVALILEGLUGLNPROLEUASNGLYPHEGLYARGLEUGLUGLUALAGLUTHRMETARGLYSGLNGLUASNGLYTHRPHEGLYASNSERGLYILEARGILEARGASPPHEGLUASNMETLYSARGASPASPALAASNPROALAASNTYRPROTYRILEVALASPTHRTYRTHRHISTYRILELEUGLUASNASNLYSVALGLUMETPHEILEASNASPLYSGLUASPSERALAPROLEULEUPROVALILEGLUASPASPARGTYRVALVALLYSTHRILEPROSERCYSARGMETSERTHRLEUGLUILEPROALAMETALAPHEHISMETPHELEUPHEGLYSERLYSLYSTHRGLULYSLEUILEVALASPVALHISASNARGTYRLYSARGLEUPHEGLNALAMETGLNLYSGLUGLUVALTHRALAGLUASNILEALASERPHEGLYILEALAGLUSERASPLEUPROGLNLYSILELEUASPLEUILESERGLYASNALAHISGLYLYSASPVALASPALAPHEILEARGLEUTHRVALASPASPMETLEUTHRASPTHRGLUARGARGILELYSARGPHELYSASPASPARGLYSSERILEARGSERALAASPASNLYSMETGLYLYSARGGLYPHELYSGLNILESERTHRGLYLYSLEUALAASPPHELEUALALYSASPILEVALLEUPHEGLNPROSERVALASNASPGLYGLUASNLYSILETHRGLYLEUASNTYRARGILEMETGLNSERALAILEALAVALTYRASPSERGLYASPASPTYRGLUALALYSGLNGLNPHELYSLEUMETPHEGLULYSALAARGLEUILEGLYLYSGLYTHRTHRGLUPROHISPROPHELEUTYRLYSVALPHEALAARGSERILEPROALAASNALAVALGLUPHETYRGLUARGTYRLEUILEGLUARGLYSPHETYRLEUTHRGLYLEUSERASNGLUILELYSLYSGLYASNARGVALASPVALPROPHEILEARGARGASPGLNASNLYSTRPLYSTHRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 7 - protein, 480 aa, synthetic construct
  REGION 1..480: note = N480 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMST

SEQ ID NO: 8 - protein, 1959 aa, synthetic construct
  REGION 1..1959: note = C481 Fragment amino acid sequence
LEUGLUILEPROALAMETALAPHEHISMETPHELEUPHEGLYSERLYSLYSTHRGLULYSLEUILEVALASPVALHISASNARGTYRLYSARGLEUPHEGLNALAMETGLNLYSGLUGLUVALTHRALAGLUASNILEALASERPHEGLYILEALAGLUSERASPLEUPROGLNLYSILELEUASPLEUILESERGLYASNALAHISGLYLYSASPVALASPALAPHEILEARGLEUTHRVALASPASPMETLEUTHRASPTHRGLUARGARGILELYSARGPHELYSASPASPARGLYSSERILEARGSERALAASPASNLYSMETGLYLYSARGGLYPHELYSGLNILESERTHRGLYLYSLEUALAASPPHELEUALALYSASPILEVALLEUPHEGLNPROSERVALASNASPGLYGLUASNLYSILETHRGLYLEUASNTYRARGILEMETGLNSERALAILEALAVALTYRASPSERGLYASPASPTYRGLUALALYSGLNGLNPHELYSLEUMETPHEGLULYSALAARGLEUILEGLYLYSGLYTHRTHRGLUPROHISPROPHELEUTYRLYSVALPHEALAARGSERILEPROALAASNALAVALGLUPHETYRGLUARGTYRLEUILEGLUARGLYSPHETYRLEUTHRGLYLEUSERASNGLUILELYSLYSGLYASNARGVALASPVALPROPHEILEARGARGASPGLNASNLYSTRPLYSTHRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 9 - protein, 550 aa, synthetic construct
  REGION 1..550: note = N550 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHG

SEQ ID NO: 10 - protein, 1749 aa, synthetic construct
  REGION 1..1749: note = C551 Fragment amino acid sequence
LYSASPVALASPALAPHEILEARGLEUTHRVALASPASPMETLEUTHRASPTHRGLUARGARGILELYSARGPHELYSASPASPARGLYSSERILEARGSERALAASPASNLYSMETGLYLYSARGGLYPHELYSGLNILESERTHRGLYLYSLEUALAASPPHELEUALALYSASPILEVALLEUPHEGLNPROSERVALASNASPGLYGLUASNLYSILETHRGLYLEUASNTYRARGILEMETGLNSERALAILEALAVALTYRASPSERGLYASPASPTYRGLUALALYSGLNGLNPHELYSLEUMETPHEGLULYSALAARGLEUILEGLYLYSGLYTHRTHRGLUPROHISPROPHELEUTYRLYSVALPHEALAARGSERILEPROALAASNALAVALGLUPHETYRGLUARGTYRLEUILEGLUARGLYSPHETYRLEUTHRGLYLEUSERASNGLUILELYSLYSGLYASNARGVALASPVALPROPHEILEARGARGASPGLNASNLYSTRPLYSTHRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 11 - protein, 624 aa, synthetic construct
  REGION 1..624: note = N624 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKI

SEQ ID NO: 12 - protein, 1527 aa, synthetic construct
  REGION 1..1527: note = C625 Fragment amino acid sequence
THRGLYLEUASNTYRARGILEMETGLNSERALAILEALAVALTYRASPSERGLYASPASPTYRGLUALALYSGLNGLNPHELYSLEUMETPHEGLULYSALAARGLEUILEGLYLYSGLYTHRTHRGLUPROHISPROPHELEUTYRLYSVALPHEALAARGSERILEPROALAASNALAVALGLUPHETYRGLUARGTYRLEUILEGLUARGLYSPHETYRLEUTHRGLYLEUSERASNGLUILELYSLYSGLYASNARGVALASPVALPROPHEILEARGARGASPGLNASNLYSTRPLYSTHRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 13 - protein, 725 aa, synthetic construct
  REGION 1..725: note = N725 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWK

SEQ ID NO: 14 - protein, 1224 aa, synthetic construct
  REGION 1..1224: note = C726 Fragment amino acid sequence
THRPROALAMETLYSTHRLEUGLYARGILETYRSERGLUASPLEUPROVALGLULEUPROARGGLNMETPHEASPASNGLUILELYSSERHISLEULYSSERLEUPROGLNMETGLUGLYILEASPPHEASNASNALAASNVALTHRTYRLEUILEALAGLUTYRMETLYSARGVALLEUASPASPASPPHEGLNTHRPHETYRGLNTRPASNARGASNTYRARGTYRMETASPMETLEULYSGLYGLUTYRASPARGLYSGLYSERLEUGLNHISCYSPHETHRSERVALGLUGLUARGGLUGLYLEUTRPLYSGLUARGALASERARGTHRGLUARGTYRARGLYSGLNALASERASNLYSILEARGSERASNARGGLNMETARGASNALASERSERGLUGLUILEGLUTHRILELEUASPLYSARGLEUSERASNSERARGASNGLUTYRGLNLYSSERGLULYSVALILEARGARGTYRARGVALGLNASPALALEULEUPHELEULEUALALYSLYSTHRLEUTHRGLULEUALAASPPHEASPGLYGLUARGPHELYSLEULYSGLUILEMETPROASPALAGLULYSGLYILELEUSERGLUILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 15 - protein, 928 aa, synthetic construct
  REGION 1..928: note = N928 Fragment amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSE

SEQ ID NO: 16 - protein, 615 aa, synthetic construct
  REGION 1..615: note = C929 Fragment amino acid sequence
ILEMETPROMETSERPHETHRPHEGLULYSGLYGLYLYSLYSTYRTHRILETHRSERGLUGLYMETLYSLEULYSASNTYRGLYASPPHEPHEVALLEUALASERASPLYSARGILEGLYASNLEULEUGLULEUVALGLYSERASPILEVALSERLYSGLUASPILEMETGLUGLUPHEASNLYSTYRASPGLNCYSARGPROGLUILESERSERILEVALPHEASNLEUGLULYSTRPALAPHEASPTHRTYRPROGLULEUSERALAARGVALASPARGGLUGLULYSVALASPPHELYSSERILELEULYSILELEULEUASNASNLYSASNILEASNLYSGLUGLNSERASPILELEUARGLYSILEARGASNALAPHEASPHISASNASNTYRPROASPLYSGLYVALVALGLUILELYSALALEUPROGLUILEALAMETSERILELYSLYSALAPHEGLYGLUTYRALAILEMETLYSGLYSERLEUGLNLEUPROPROLEUGLUARGLEUTHRLEUGLYSERSERTYRPROTYRASPVALPROASPTYRALATYRPROTYRASPVALPROASPTYRALATYRPROTYRASPVALPROASPTYRALA

SEQ ID NO: 17 - protein, 93 aa, synthetic construct [sequence omitted]

SEQ ID NO: 18 - protein, 107 aa, synthetic construct
  REGION 1..107: note = FKBP amino acid sequence
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 19 - protein, 150 aa, synthetic construct
  REGION 1..150: note = nMag amino acid sequence
HTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 20 - protein, 150 aa, synthetic construct
  REGION 1..150: note = pMag amino acid sequence
HTLYAPGGYDIMGYLRQIRNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETE

SEQ ID NO: 21 - protein, 32 aa, synthetic construct
  REGION 1..32: note = linker
GSGSGSGSGSGSGSGSGSGSGSGSGSGSGSGS

SEQ ID NO: 22 - DNA, 816 bp, synthetic construct
  misc_feature 1..816: note = N272 Fragment polynucleotide sequence
  source 1..816: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaac

SEQ ID NO: 23 - DNA, 2586 bp, synthetic construct
  misc_feature 1..2586: note = C273 Fragment polynucleotide sequence
  source 1..2586: mol_type = other DNA
agcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 24 - DNA, 858 bp, synthetic construct
  misc_feature 1..858: note = N286 Fragment polynucleotide sequence
  source 1..858: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtcc

SEQ ID NO: 25 - DNA, 2544 bp, synthetic construct
  misc_feature 1..2544: note = C287 Fragment polynucleotide sequence
  source 1..2544: mol_type = other DNA
aacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 26 - DNA, 1053 bp, synthetic construct
  misc_feature 1..1053: note = N351 Fragment polynucleotide sequence
  source 1..1053: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgac

SEQ ID NO: 27 - DNA, 2349 bp, synthetic construct
  misc_feature 1..2349: note = C352 Fragment polynucleotide sequence
  source 1..2349: mol_type = other DNA
cacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 28 - DNA, 1440 bp, synthetic construct
  misc_feature 1..1440: note = N480 Fragment polynucleotide sequence
  source 1..1440: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcacc

SEQ ID NO: 29 - DNA, 1962 bp, synthetic construct
  misc_feature 1..1962: note = C481 Fragment polynucleotide sequence
  source 1..1962: mol_type = other DNA
ctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 30 - DNA, 1650 bp, synthetic construct
  misc_feature 1..1650: note = N550 Fragment polynucleotide sequence
  source 1..1650: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggc

SEQ ID NO: 31 - DNA, 1752 bp, synthetic construct
  misc_feature 1..1752: note = C551 Fragment polynucleotide sequence
  source 1..1752: mol_type = other DNA
aaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 32 - DNA, 1872 bp, synthetic construct
  misc_feature 1..1872: note = N624 Fragment polynucleotide sequence
  source 1..1872: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatc

SEQ ID NO: 33 - DNA, 1530 bp, synthetic construct
  misc_feature 1..1530: note = C625 Fragment polynucleotide sequence
  source 1..1530: mol_type = other DNA
accggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 34 - DNA, 2175 bp, synthetic construct
  misc_feature 1..2175: note = N725 Fragment polynucleotide sequence
  source 1..2175: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaa

SEQ ID NO: 35 - DNA, 1227 bp, synthetic construct
  misc_feature 1..1227: note = C726 Fragment polynucleotide sequence
  source 1..1227: mol_type = other DNA
acacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 36 - DNA, 2784 bp, synthetic construct
  misc_feature 1..2784: note = N928 Fragment polynucleotide sequence
  source 1..2784: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgag

SEQ ID NO: 37 - DNA, 618 bp, synthetic construct
  misc_feature 1..618: note = C929 Fragment polynucleotide sequence
  source 1..618: mol_type = other DNA
atcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 38 - DNA, 279 bp, synthetic construct
  misc_feature 1..279: note = FRB polynucleotide sequence
  source 1..279: mol_type = other DNA
atcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaag

SEQ ID NO: 39 - DNA, 321 bp, synthetic construct
  misc_feature 1..321: note = FKBP polynucleotide sequence
  source 1..321: mol_type = other DNA
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 40 - DNA, 450 bp, synthetic construct
  misc_feature 1..450: note = nMag polynucleotide sequence
  source 1..450: mol_type = other DNA
cacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgag

SEQ ID NO: 41 - DNA, 450 bp, synthetic construct
  misc_feature 1..450: note = pMag polynucleotide sequence
  source 1..450: mol_type = other DNA
cacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgag

SEQ ID NO: 42 - DNA, 1206 bp, synthetic construct
  misc_feature 1..1206: note = N272-GS16-FRB-GS16 polynucleotide sequence
  source 1..1206: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacaacccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 43 - DNA, 3012 bp, synthetic construct
  misc_feature 1..3012: note = GS16-FKBP-GS16-C273 polynucleotide sequence
  source 1..3012: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacaagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 44 - DNA, 1248 bp, synthetic construct
  misc_feature 1..1248: note = N286-GS16-FRB-GS16 polynucleotide sequence
  source 1..1248: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 45 - DNA, 2970 bp, synthetic construct
  misc_feature 1..2970: note = GS16-FKBP-GS16-C287 polynucleotide sequence
  source 1..2970: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacaaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 46 - DNA, 1443 bp, synthetic construct
  misc_feature 1..1443: note = N351-GS16-FRB-GS16 polynucleotide sequence
  source 1..1443: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccttccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 47 - DNA, 2775 bp, synthetic construct
  misc_feature 1..2775: note = GS16-FKBP-GS16-C352 polynucleotide sequence
  source 1..2775: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacacacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 48 - DNA, 1830 bp, synthetic construct
  misc_feature 1..1830: note = N480-GS16-FRB-GS16 polynucleotide sequence
  source 1..1830: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcacccttccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 49 - DNA, 2388 bp, synthetic construct
  misc_feature 1..2388: note = GS16-FKBP-GS16-C481 polynucleotide sequence
  source 1..2388: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacactggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 50 - DNA, 2040 bp, synthetic construct
  misc_feature 1..2040: note = N550-GS16-FRB-GS16 polynucleotide sequence
  source 1..2040: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcttcccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 51 - DNA, 2178 bp, synthetic construct
  misc_feature 1..2178: note = GS16-FKBP-GS16-C551 polynucleotide sequence
  source 1..2178: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacaaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 52 - DNA, 2262 bp, synthetic construct
  misc_feature 1..2262: note = N624-GS16-FRB-GS16 polynucleotide sequence
  source 1..2262: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaacccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 53 - DNA, 1956 bp, synthetic construct
  misc_feature 1..1956: note = GS16-FKBP-GS16-C625 polynucleotide sequence
  source 1..1956: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacaaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 54 - DNA, 2565 bp, synthetic construct
  misc_feature 1..2565: note = N725-GS16-FRB-GS16 polynucleotide sequence
  source 1..2565: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaacccccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 55 - DNA, 1653 bp, synthetic construct
  misc_feature 1..1653: note = GS16-FKBP-GS16-C726 polynucleotide sequence
  source 1..1653: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 56 - DNA, 3174 bp, synthetic construct
  misc_feature 1..3174: note = N928-GS16-FRB-GS16 polynucleotide sequence
  source 1..3174: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagttcccgggatccggaggaagtggcagctctggcggcagtggagggtctggtggcagcggaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagggaggaagcggcggttccggggggggatccggaagtggttccggggggtag

SEQ ID NO: 57 - DNA, 1044 bp, synthetic construct
  misc_feature 1..1044: note = GS16-FKBP-GS16-C929 polynucleotide sequence
  source 1..1044: mol_type = other DNA
atgggaggaagtggcagctctggcggcagtggagggtctggtggcagcggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggaggaagcggcggttccggggggggatccggaagtggttccggggggtgtacaatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 58 - DNA, 1377 bp, synthetic construct
  misc_feature 1..1377: note = N272-GS16-pMag-GS16 polynucleotide sequence
  source 1..1377: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacaacccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 59 - DNA, 3141 bp, synthetic construct
  misc_feature 1..3141: note = GS16-nMag-GS16-C273 polynucleotide sequence
  source 1..3141: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacaagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 60 - DNA, 1419 bp, synthetic construct
  misc_feature 1..1419: note = N286-GS16- pMag -GS16 polynucleotide sequence
  source 1..1419: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 61 - DNA, 3099 bp, synthetic construct
  misc_feature 1..3099: note = GS16- nMag -GS16-C287 polynucleotide sequence
  source 1..3099: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacaaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 62 - DNA, 1614 bp, synthetic construct
  misc_feature 1..1614: note = N351-GS16- pMag -GS16 polynucleotide sequence
  source 1..1614: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccttccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 63 - DNA, 2904 bp, synthetic construct
  misc_feature 1..2904: note = GS16- nMag -GS16-C352 polynucleotide sequence
  source 1..2904: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacacacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 64 - DNA, 2001 bp, synthetic construct
  misc_feature 1..2001: note = N480-GS16- pMag -GS16 polynucleotide sequence
  source 1..2001: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcacccttccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 65 - DNA, 2517 bp, synthetic construct
  misc_feature 1..2517: note = GS16- nMag -GS16-C481 polynucleotide sequence
  source 1..2517: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacactggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 66 - DNA, 2211 bp, synthetic construct
  misc_feature 1..2211: note = N550-GS16- pMag -GS16 polynucleotide sequence
  source 1..2211: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcttcccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 67 - DNA, 2307 bp, synthetic construct
  misc_feature 1..2307: note = GS16- nMag -GS16-C551 polynucleotide sequence
  source 1..2307: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacaaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 68 - DNA, 2433 bp, synthetic construct
  misc_feature 1..2433: note = N624-GS16- pMag -GS16 polynucleotide sequence
  source 1..2433: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaacccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 69 - DNA, 2085 bp, synthetic construct
  misc_feature 1..2085: note = GS16- nMag -GS16-C625 polynucleotide sequence
  source 1..2085: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacaaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 70 - DNA, 2736 bp, synthetic construct
  misc_feature 1..2736: note = N725-GS16- pMag -GS16 polynucleotide sequence
  source 1..2736: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaacccccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 71 - DNA, 1782 bp, synthetic construct
  misc_feature 1..1782: note = GS16- nMag -GS16-C726 polynucleotide sequence
  source 1..1782: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 72 - DNA, 3345 bp, synthetic construct
  misc_feature 1..3345: note = N928-GS16- pMag -GS16 polynucleotide sequence
  source 1..3345: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagttcccgggatccggaggaagtggcagctctggcggcagtggagggtctggtggcagcggacacaccctgtacgcccccggcggctacgacatcatgggctacctgagacagatcagaaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgagggaggaagcggcggttccggggggggatccggaagtggttccggggggtag

SEQ ID NO: 73 - DNA, 1173 bp, synthetic construct
  misc_feature 1..1173: note = GS16- nMag -GS16-C929 polynucleotide sequence
  source 1..1173: mol_type = other DNA
atgggaggaagtggcagctctggcggcagtggagggtctggtggcagcggacacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatgagaaaggccatcgacagaaacgccgaggtgcaggtggaggtggtgaacttcaagaagaacggccagagattcgtgaacttcctgaccatgatccccgtgagagacgagaccggcgagtacagatacagcatgggcttccagtgcgagaccgagggaggaagcggcggttccggggggggatccggaagtggttccggggggtgtacaatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaa

SEQ ID NO: 74 - protein, 150 aa, synthetic construct
  REGION 1..150: note = nMagHigh1 amino acid sequence
HTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETE

SEQ ID NO: 75 - DNA, 450 bp, synthetic construct
  misc_feature 1..450: note = nMagHigh1 polynucleotide sequence
  source 1..450: mol_type = other DNA
cacaccctgtacgcccccggcggctacgacatcatgggctacctggaccagatcggcaacagacccaacccccaggtggagctgggccccgtggacaccagctgcgccctgatcctgtgcgacctgaagcagaaggacacccccatcgtgtacgccagcgaggccttcctgtatatgaccggctacagcaacgccgaggtgctgggcagaaactgcagattcctgcagagccccgacggcatggtgaagcccaagagcaccagaaagtacgtggacagcaacaccatcaacaccatcagaaaggccatcgacagaaacgccgaggtgcaggtggaggtggtgaacttcaagaagaacggccagagattcgtgaacttcctgaccatcatccccgtgagagacgagaccggcgagtacagatacagcatgggcttccagtgcgagaccgag

(19) United States
(12) Patent Application Publication
(10) Pub. No.: US 2024/0309343 A1
(43) Pub. Date: Sep. 19, 2024

(54) RNA expression modulating or editing method via regulation of Cas13 protein

(71) Applicant: Korea Advanced Institute of Science and Technology, Daejeon (KR)

(72) Inventors: Won Do Heo, Daejeon (KR); Jeonghye Yu, Daejeon (KR)

(21) Appl. No.: 17/907,472

(22) PCT Filed: Jul. 8, 2022
(86) PCT No.: PCT/KR2022/009993

§ 371 Date: Sep. 27, 2022

(30) Foreign Application Priority Data
Jul. 8, 2021 (KR) 10-2021-0089435
Apr. 20, 2022 (KR) 10-2022-0048556

(51) Int. Cl. C12N 9/22; C12N 9/86; C12N 13/00; C12N 15/11; C12N 15/85

(52) U.S. Cl. CPC C12N 9/22; C12N 9/86; C12N 13/00; C12N 15/111; C12N 15/85; C12N 2310/20; C12Y 305/04004

(57) ABSTRACT

The present invention relates to a RNA expression modulating or editing method through chemical or optogenetic regulation of Cas13 protein activity. Specifically, in order to regulate the activity of Cas13 in the CRISPR-Cas13 system, a fragment of the Cas13 protein was generated to enable recombination, and a chemogenetically or optogenetically bindable protein was linked to each fragment. And, it was confirmed that the Cas13 protein can be activated by treatment of a low molecular weight compound or irradiation of light. Accordingly, the method of the present invention can be effectively used for disease treatment by regulating RNA expression or editing RNA mutations related to disease.

### BACKGROUND OF THE INVENTION

### 1. Field of the Invention

The present invention relates to a RNA expression modulating or editing method through chemical or optogenetic regulation of Cas13 protein activity.

### 2. Description of the Related Art

Clustered Regularly Interspaced Short Palindromic Repeats (CRISPR), in combination with associated sequences (cas), constitute CRISPR-Cas system, which confers adaptive immunity in many bacteria. The CRISPR-Cas system consists of short DNA repeats interposed by hypervariable sequences that provide adaptive immunity against invasive genetic elements such as phages and plasmids through sequence-specific targeting and interference. Typically, an invasive DNA sequence is acquired as a new “spacer”, each paired with a CRISPR repeat and inserted as a new repeat-spacer unit at the CRISPR gene locus. The repeat-spacer array is then transcribed into long pre-CRISPR RNA (pre-crRNA), which is processed into small interfering CRISPR RNAs (crRNA) that induce sequence-specific recognition. In particular, crRNA induces nucleases for sequence-specific nucleic acid cleavage mediated by Cas endonucleases to complementary targets. The CRISPR-Cas system is identified in half of bacteria (˜46%) and the majority of archaea (˜90%).

The Cas system is currently being applied to many therapeutic fields by editing DNA sequence by substituting an appropriate guide molecule by targeting cellular DNA. In addition to Cas9 system capable of targeting DNA, several Cas proteins have been discovered, among which Cas13 protein targets single strand RNA.

The CRISPR-Cas13 system was discovered as a system that targets RNA differently from Cas9, and was first reported as C2c2 in 2016. The existing CRISPR-Cas13 system is mainly composed of four subtypes, which are characterized by having two HEPN domains (higher eukaryotes and prokaryotes nucleotide-binding domains) with RNase activity. Cas13, similar to Cas9, forms a complex with crRNA to cleave the target ssRNA, rendering the foreign gene inoperable.

The CRISPR-Cas13 system has been applied to various fields so far. There are literatures disclosing that Cas13a expression can be induced by inserting a promoter capable of binding to NF-κB, which is overexpressed in cancer (Molecular Therapy: Oncolytics Vol. 19 Dec. 2020), disclosing that the cleavage efficiency is different for each spacer of crRNA in glioma cells (Adv. Sci. 2019, 6, 1901299), applying the CRISPR-Cas13 system as a RNA virus therapeutic agent (Molecular Therapy: Nucleic Acids Vol. 19 Mar. 2020), and disclosing the K-RAS gene-specific spacer-specific cleavage of only the mutant K-Ras of cancer cells (Cancer Letters, Volume 431, 1 Sep. 2018, Pages 171-181). In the case of neurodegenerative diseases such as Parkinson's disease, the number of neurons is reduced. There have been reports in the literatures disclosing the possibility of treating neurological diseases with a glia-to-neuron transformation system through Cas13d transmission (Cell, Volume 181, Issue 3, 30 Apr. 2020, Pages 590-603.), and disclosing that the CRISPR-Cas system is effectively applied in zebrafish (Developmental Cell, Volume 54, Issue 6, 28 Sep. 2020, Pages 805-817.). However, in the case of the Cas13 system, there is a limitation in that expression cannot be controlled.

Optogenetics is a biological technology that can control cells in living tissues with light, and a typical example is the expression of ion channels that respond to light by genetically manipulating nerve cells. Optogenetics is a technology that combines optics and genetics, and by using it, it is possible to control and observe the activity of individual nerve cells in living tissues, even in freely moving animals, and to check in real time what effect the regulation of nerve activity causes. The main material required for optogenetics is a protein that responds to light. In order to control neural activity, optogenetic effectors such as channelrhodopsin, halorhodopsin and archaerhodopsin are used. To record neural activity optically, optogenetic sensors such as GCaMP detecting changes in calcium concentration, synaptopHluorin detecting secretion of neural endoplasmic reticulum, GluSnFRs detecting neurotransmitters, and Arclightning (ASAP1) detecting cell membrane potential are used. By using optogenetics, the neural activity of genetically classified specific neurons can be selectively controlled or recorded, and the target location and time can precisely controlled by using light. Therefore, the use of optogenetics has the potential to well regulate the CRISPR-Cas13 system in cells through intensity, temporal, and spatial control.

In addition, chemical genetics is a powerful method that uses small molecule regulators to inform the molecular basis of various biological processes, and refers to the control of proteins by perturbing cellular or physiological functions using low molecular weight compounds. As an example of regulating the function of a protein with a low molecular compound by chemical genetics, it is disclosed that a fusion protein comprising the heterodimer-forming proteins FRB and FKBP can form various oligomers according to addition of rapamycin (J BiosciBioeng. 2016 July; 122 (1): 40-6.).

Accordingly, the present inventors generated a fragment of Cas13 protein to enable recombination in order to regulate the activity of Cas13 in the CRISPR-Cas13 system, linked a chemogenetically or optogenetically bindable protein to each fragment, and the present inventors completed the present invention by confirming that the Cas13 protein could be activated by treatment of a low molecular weight compound or irradiation of light.

### SUMMARY OF THE INVENTION

It is an object of the present invention to provide a RNA expression modulating or editing method through chemical or optogenetic regulation of Cas13 protein activity.

To achieve the above object, the present invention provides a method for regulating the activity of Cas13 protein, comprising a step of treating a first fragment containing the N-terminal segment of Cas13 protein and a first regulatory protein; and a second fragment containing a second regulatory protein and the C- terminal segment of Cas13 protein with a compound or light irradiation.

The present invention also provides a method for modulating the expression of a target RNA comprising the following steps:
1) preparing a vector comprising a construct containing a sequence encoding the N-terminal segment of Cas13 protein and a sequence encoding a first regulatory protein; and a vector comprising a construct containing a second fragment comprising a sequence encoding a second regulatory protein and a sequence encoding the C-terminal segment of Cas13 protein;
2) injecting the vector above and crRNA (CRISPR RNA) capable of hybridizing to the target RNA sequence into mammalian cells; and
3) treating with a compound or irradiating with light.

The present invention also provides a method for editing the target RNA sequence comprising the following steps:
1) preparing a vector comprising a construct containing a sequence encoding the N-terminal segment of Cas13 protein and a sequence encoding a first regulatory protein; and a vector comprising a construct containing a second fragment comprising a sequence encoding a second regulatory protein, a sequence encoding the C-terminal segment of Cas13 protein and a sequence encoding a RNA editing protein;
2) injecting the vector above and crRNA (CRISPR RNA) capable of hybridizing to the target RNA sequence into mammalian cells; and
3) treating with a compound or irradiating with light.

### Advantageous Effect

The present invention relates to a RNA expression modulating or editing method through chemical or optogenetic regulation of Cas13 protein activity. Specifically, in order to regulate the activity of Cas13 in the CRISPR-Cas13 system, a fragment of the Cas13 protein was generated to enable recombination, and a chemogenetically or optogenetically bindable protein was linked to each fragment. And, it was confirmed that the Cas13 protein can be activated by treatment of a low molecular weight compound or irradiation of light. Accordingly, the method of the present invention can be effectively used for disease treatment by regulating RNA expression or editing RNA mutations related to disease.

### BRIEF DESCRIPTION OF THE DRAWINGS

FIG. 1 is a schematic diagram showing the structure of the predicted PspCas13b protein, in which eight Cas13b fragment candidates are indicated.

FIG. 2 is a schematic diagram showing the cleavage sites of a Cas13b fragment candidate that is expected to exhibit Cas13b activity by conserving two HEPN domains having RNase activity.

FIG. 3 is a schematic diagram showing the construct in which each regulatory protein is connected with a linker (GS16) so as not to affect Cas13b protein function using the FKBP (FK506-binding protein)-FRB (FKBP-rapamycin-binding) system, which is known to be dimerized by rapamycin.

FIG. 4 is a schematic diagram showing the construct in which each regulatory protein is connected with a linker (GS16) using the nMag (negative Mag)-pMag (positive Mag) system, which is known to be dimerized by blue light.

FIG. 5 is a graph showing the Cas13 activity measured by double luciferase analysis after treatment with 500 nM rapamycin (error bar, mean±sem, n=5, student's two-tailed t-test).

FIG. 6 is a schematic diagram showing the crRNA-mediated RNA degradation activity of Cas13 to allow the Cas13 fragment cleaved by rapamycin treatment inducing heterodimerization between FRB and FKBP to recombine.

FIG. 7 is a graph showing the results of the second screening of the Cas13 cleavage site near the residues 351 and 352 (error bar, mean±sem, n=3, student's two-tailed t-test).

FIG. 8 is a schematic diagram showing the crRNA-mediated RNA degradation activity of Cas13 (paCas13) to allow the Cas13 fragment cleaved by blue light treatment inducing heterodimerization between pMag and nMag to recombine.

FIG. 9 is a set of graphs showing the decrease in luciferase activity upon light stimulation using the paCas13 fragment fused with photoinducible dimerization domains (pMag:nMag and pMag:nMagHigh1).

FIG. 10 is a schematic diagram showing the paCas13 candidates based on Cas13 cleavage sites near the residues 351 and 352.

FIG. 11 is a set of graphs showing the light-induced RNA knockdown of endogenous KRAS, PPIB and STAT3 in HEK293T cells transfected with paCas13-1, paCas13-2 or paCas13-3 and multiple crRNAs (error bar, mean±sem, n=3).

FIG. 12 is a schematic diagram showing the modulated RNA regulation by coupling padCas13 (including H133A and H1058A mutations that inactivate the HEPN RNase domain of Cas13b) with ADAR2DD.

FIG. 13 is a schematic diagram showing the A-to-I RNA editing reporter assay, in which a single G-to-A mutation is introduced into the firefly luciferase coding sequence, resulting in W417X (X=STOP) codon switch, a measurable firefly luciferase signal is lost.

FIG. 14 is a set of graphs showing the analysis results over time of the padCas13 editor under the light conditions quantified by the restoration of the firefly luciferase signal (D: dark, L: light).

FIG. 15 is a graph showing the standard curve for correcting Sanger sequencing chromatogram with fluorophore and PCR bias.

FIG. 16 is a diagram showing the sequencing chromatogram under the dark (top) or light (bottom) condition.

FIG. 17 is a set of graphs showing the RNA editing percentage quantified and normalized by RT-PCR-Sanger sequencing (D: dark, L: light).

FIG. 18 is a graph showing the RNA editing percentage of transfected cells having only crRNA by RT-PCR-Sanger sequencing.

FIG. 19 is a graph showing the deep-sequencing quantification (n=1) of A-to-I RNA editing by the padCas13 editor under the light stimulation for 24 hours.

FIG. 20 is a diagram showing the results of RNA-seq reading surrounding the target site (1250A→I) of Firefly luciferase editing.

FIG. 21 is a schematic diagram showing the EGFP indicator for the light-induced C-to-U RNA editing activity.

FIG. 22 is a diagram showing the representative fluorescence microscopy images of HEK293T cells co-transfected with the padCas13-2 editor, EGFP indicator and target crRNA.

FIG. 23 is a set of graphs showing the representative flow cytometry plots transfected with the padCas13-2 editor [X-axis: FITC-A (log scale), Y-axis: SSC-A (linear scale)].

FIG. 24 is a graph showing the quantification of EGFP H66Y restored by the padCas13 editor.

FIG. 25 is a graph showing the A-to-I RNA editing percentage by the padCas13-1 or padCas13-2 editor under the light stimulation for 24 hours (error bar, mean±sem, n=3).

FIG. 26 is a graph showing the C-to-U RNA editing percentage by the padCas13-1 or padCas13-2 editor under the light stimulation for 24 hours (error bar, mean±sem, n=3).

### DESCRIPTION OF THE PREFERRED EMBODIMENTS

Hereinafter, the present invention is described in detail.

The present invention provides a method for regulating the activity of Cas13 protein, comprising a step of treating a first fragment containing the N-terminal segment of Cas13 protein and a first regulatory protein; and a second fragment containing a second regulatory protein and the C-terminal segment of Cas13 protein with a compound or light irradiation.

The Cas13 protein is a kind of Cas protein. Cas protein is CRISPR-related protein, and is an enzyme that can recognize and cut nucleic acids such as DNA or RNA when they are double-stranded or single-stranded (dsDNA/RNA and ssDNA/RNA). Specifically, Cas protein can recognize and cut a double strand or single strand nucleic acid bound to crRNA or guide RNA. That is, the endonuclease function of Cas protein is activated by recognizing that crRNA is bound to the target site. In addition, Cas protein may have exonuclease activity capable of non-specific cleaving of double strand and/or single strand DNA and/or RNA as its endonuclease function is activated.

The Cas13 protein can be any one protein selected from the group consisting of Cas13a, Cas13b, Cas13c and Cas13d, and can naturally recognize and cut ‘RNA’, and is known as “C2c2” in bacteria. Cas13 is a class 2, type VI CRISPR protein, and is activated by recognizing an ssRNA target. Cas13a found in Leptotrichiawadei and Cas13b found in Prevotella sp. P5-125 are representative, both of which do not require a specific motif such as PAM.

The Cas13b protein is associated with one or more functional domains, and the effector protein contains one or more mutations in the HEPN domain so that the complex can transmit epigenetic modifiers or transcriptional or translational activation or repression signals. The complex can be formed in vitro or ex vivo, introduced into cells or contacted with RNA; or formed in vivo.

The Cas13b protein is not particularly limited, but in a specific embodiment of the present invention, the Cas13b protein is PspCas13b.

The PspCas13b protein is the most expressed in animal cells and has been proven to have an effective KD effect. The structure of the PspCas13b protein consists of two HEPN domains having RNase activity, crRNA and a domain that interacts with the target RNA.

A linker can be inserted between the N-terminal segment of Cas13 protein and the first regulatory protein, and between the second regulatory protein and the C-terminal segment of Cas13 protein.

The linker includes glycine (G) and serine (S), and can have an amino acid sequence in which glycine and serine are repeated 16 times (GSGSGSGSGSGSGSGSGSGSGSGSGSGSGSGS, SEQ. ID. NO: 21).

The N-terminal segment of Cas13 protein can be a segment from the N-terminus of Cas13 protein to the 272^{th} (SEQ. ID. NO: 1), 286^{th} (SEQ. ID. NO: 3), 351^{st} (SEQ. ID. NO: 5), 480^{th} (SEQ. ID. NO: 7), 550^{th} (SEQ. ID. NO: 9), 624^{th} (SEQ. ID. NO: 11), 725^{th} (SEQ. ID. NO: 13) or 928^{th} (SEQ. ID. NO: 15) amino acid, and the segment includes one HEPN domain having RNase activity.

The C-terminal segment of Cas13 protein can be a segment from the C-terminus of Cas13 protein to the 861^{th} (SEQ. ID. NO: 2), 847^{th} (SEQ. ID. NO: 4), 782^{th} (SEQ. ID. NO: 6), 653^{th} (SEQ. ID. NO: 8), 583^{th} (SEQ. ID. NO: 10), 509^{th} (SEQ. ID. NO: 12), 408^{th} (SEQ. ID. NO: 14) or 205^{th} (SEQ. ID. NO: 16) amino acid, and the segment includes one HEPN domain having RNase activity.

The compound treatment or light irradiation induces dimerization of the first regulatory protein and the second regulatory protein.

The compound treatment refers to chemogenetically control by disturbing the cellular or physiological function of a protein using a low molecular weight compound.

The compound is not particularly limited, but in a specific embodiment of the present invention, the compound is rapamycin.

The light irradiation refers to a biological technology capable of controlling cells of a living tissue with light.

The light is not particularly limited, but in a specific embodiment of the present invention, the light is blue light.

The combination of the first regulatory protein and the second regulatory protein is not particularly limited, but in a specific embodiment of the present invention, the combination of the first regulatory protein and the second regulatory protein is FKBP (SEQ. ID. NO: 18)—FRB (SEQ. ID. NO: 17), nMag (SEQ. ID. NO: 19)—pMag (SEQ. ID. NO: 20) or nMagH (SEQ. ID. NO: 74)—pMag (SEQ. ID. NO: 20).

Rapamycin, an antifungal antibiotic, mediates the tight heterodimer formation by simultaneously binding to the 12-kDa FK506 binding protein (FKBP) and FKBP rapamycin binding (FRB) domains (K_{d}=2.5 nM). Various cellular functions such as gene expression and protein translocation can be artificially controlled by conditional dimerization of a protein of interest fused to FKBP or FRB by adding rapamycin.

The said nMag-pMag was designed to recognize each other based on electrostatic interaction, preventing homodimerization and causing heterodimerization induced by light irradiation. The nMag-pMag system has the advantage that it has a small size similar to that of FKBP-FRB and can control the dissociation reaction rate. In nMagH (nMagHigh1), the 135^{th} methionine (M) and the 165^{th} methionine of nMag are mutated to isoleucine (I).

The present invention also provides a method for modulating the expression of a target RNA comprising the following steps:
1) preparing a vector comprising a construct containing a sequence encoding the N-terminal segment of Cas13 protein and a sequence encoding a first regulatory protein; and a vector comprising a construct containing a second fragment comprising a sequence encoding a second regulatory protein and a sequence encoding the C-terminal segment of Cas13 protein;
2) injecting the vector above and crRNA (CRISPR RNA) capable of hybridizing to the target RNA sequence into mammalian cells; and
3) treating with a compound or irradiating with light.

The mammalian cells can be isolated human cells or non-human mammalian cells.

The crRNA includes a guide sequence capable of hybridizing to the target RNA sequence and a direct repeat sequence.

The crRNA is CRISPR RNA, and can be single strand RNA. In addition, the crRNA can bind to tracrRNA to actuate CRISPR-associated protein, and the crRNA can be used in combination with tracrRNA. At this time, the CRNA can have a sequence complementary to a gene sequence specifically present in the target.

The crRNA can be RNA composed of 15 to 40 nucleic acids. At this time, the polynucleotide can be composed of 18 to 30 or 20 to 25 nucleic acids. In one embodiment, the crRNA may consist of 20 nucleic acids. In addition, the crRNA can include an additional sequence at 3′ to make the CRISPR-associated protein active.

The crRNA comprises a spacer sequence and a direct repeat (DR) sequence or a derivative thereof. In a preferred embodiment of the present invention, the spacer sequence or a derivative thereof comprises a seed sequence, and the seed sequence is important for recognition and/or hybridization to a sequence at the target locus. In a preferred embodiment of the present invention, the crRNA is a short crRNA capable of being associated with a short DR sequence.

The crRNA comprises a bulge, hairpin or stem loop structure. The loop is preferably GAAA, but is not limited to this sequence, and in fact is not limited to only 4 bp in length. Indeed, the preferred loop-forming sequence used in the hairpin structure is 4 nucleotides in length and most preferably has the sequence of GAAA. However, longer or shorter loop sequences can be used, and surrogate sequences can also be used. The sequence preferably comprises a nucleotide triple (for example, AAA), and an additional nucleotide (for example, C or G). Examples of loop forming sequences include CAAA and AAAG.

The crRNA may be chemically modified. Examples of chemical modifications include the inclusion of 2′-O-methyl (M), 2′-O-methyl 3′-phosphorothioate (MS) or 2′-O-methyl 3′-thio PACE (MSP) at one or more terminal nucleotides, but not always limited thereto. Such chemically modified crRNA may have increased stability and increased activity compared to unmodified crRNA, although the on-target versus off-target specificity is not predictable.

The chemically modified crRNA includes, without limitation, RNA having locked nucleic acid (LNA) nucleotides comprising a phosphorothioate linkage and a methylene bridge between the 2′ and 4′ carbons of a ribose ring.

A vector can be used for synthesizing a polynucleotide encoding Cas13b protein and crRNA used in the method of the present invention, or for modulating the expression of a target RNA or editing the target RNA. As used herein, a “vector” is a tool that allows or facilitates the transfer of an entity from one environment to another environment. A replicon such as plasmid, phage or cosmid, or another DNA segment can be inserted into the vector, causing replication of the inserted segment. In general, a vector is capable of replication when associated with appropriate control element.

In general, the term “vector” refers to a nucleic acid molecule capable of delivering another nucleic acid to which it has been linked. A vectors includes, without limitation, nucleic acid molecules that are single-stranded, double-stranded or partially double-stranded; nucleic acid molecules containing one or more free ends, or not containing free ends (for example: circular); nucleic acid molecules containing DNA, RNA or both; and other types of polynucleotides known in the art. One type of vector is a “plasmid”, which refers to a circular double-stranded DNA loop into which additional DNA fragments can be inserted, for example, by standard molecular cloning techniques. Another type of vector is a viral vector, in which a virus-derived DNA or RNA sequence is present in a vector that is encapsulated in a virus (for example, retrovirus, replication deficient retrovirus, adenovirus, replication deficient adenovirus and adeno-associated virus (AAV)). A viral vector also includes a polynucleotide carried by a virus for transfection into host cells. Certain vectors (for example, bacterial vectors and episomal mammalian vectors having a bacterial origin of replication) are capable of autonomous replication in the host cell into which they are introduced. Other vectors (for example, non-episomal mammalian vectors) integrate into the genome of the host cell upon introduction into the host cell, thereby being replicated along with the host genome. Moreover, certain vectors are capable of directing the expression of genes to which they are operably linked. Such vectors are referred to herein as “expression vectors”. Conventional expression vectors useful in recombinant DNA technology often exist in the form of plasmids. A recombinant expression vector can contain the nucleic acid of the present invention in a form suitable for expression of the nucleic acid in a host cell, which means that the recombinant expression vector contains one or more regulatory elements. The one or more regulatory elements can be selected based on the host cell to be used for the expression and is operably linked to the nucleic acid sequence to be expressed. In the recombinant expression vector, “operably linked” means that the target nucleotide sequence (for example, in an in vitro transcription/translation system, or in a host cell if the vector is introduced into the host cell) is linked to the regulatory elements in a manner that enables expression of the nucleotide sequence.

The vector can be any one selected from the group consisting of plasmids and viruses.

Specific examples of the plasmid DNA include commercial plasmids such as pCMV3, pET28a, pUC57 and pET. Other examples of the plasmid that can be used in the present invention include E. coli-derived plasmids (pUC57, pCMV3, pET28a, pET, pGEX, pQE, pDEST and pCOLD), Bacillus subtilis-derived plasmids (pUB110 and pTP5) and yeast-derived plasmids (YEp13, YEp24 and YCp50). Since these plasmids show different protein expression levels and modifications depending on the host cell, a host cell most suitable for the purpose can be selected and used.

Specific examples of the virus include adeno-associated virus (AAV), adenovirus, retrovirus, lentivirus, modified vaccinia virus ankara (MVA), herpes simplex virus or baculovirus.

In conducting any of the methods disclosed herein, a suitable vector can be introduced into a cell or embryo via one or more methods known in the art, including microinjection, electroporation, sonoporation, biolistics, calcium phosphate-mediated transfection, cation transfection, liposome transfection, dendrimer transfection, heat shock transfection, nucleofection transfection Induction, magnetofection, lipofection, impairfection, optical transfection, dedicated agent-enhanced uptake of nucleic acids, and delivery via liposomes, immunoliposomes, virosomes or artificial virions.

In some methods, a vector is introduced into a cell by microinjection. A vector or vectors can be microinjected into the nucleus or cytoplasm. In some methods, a vector or vectors can be introduced into a cell by nucleofection.

A vector can be designed for expression of CRISPR transcript (for example, nucleic acid transcript, protein or enzyme) in prokaryotic or eukaryotic cells. For example, the CRISPR transcript can be expressed in bacterial cells such as Escherichia coli, insect cells (using a baculovirus expression vector), yeast cells, or mammalian cells.

A recombinant expression vector can be transcribed and translated in vitro using, for example, a T7 promoter regulatory sequence and T7 polymerase.

The Cas13 protein forms a complex with the crRNA. The complex can be in the form of a ribonucleoprotein (RNP), and can be passed through a cell membrane in the form of RNP and delivered into a cell or a living body. In addition, the complex can be delivered into a cell or a living body by being included in a conventional RNA carrier.

The present invention also provides a method for editing the target RNA sequence comprising the following steps:
1) preparing a vector comprising a construct containing a sequence encoding the N-terminal segment of Cas13 protein and a sequence encoding a first regulatory protein; and a vector comprising a construct containing a second fragment comprising a sequence encoding a second regulatory protein, a sequence encoding the C-terminal segment of Cas13 protein and a sequence encoding a RNA editing protein;
2) injecting the vector above and crRNA (CRISPR RNA) capable of hybridizing to the target RNA sequence into mammalian cells; and
3) treating with a compound or irradiating with light.

The Cas13 protein can be deadCas13 protein from which RNase activity is removed.

The deadCas13 protein is inactivated by mutating two histidines of HEPN to alanines to prevent the HEPN domain having RNase activity from functioning.

The RNA editing protein can be ADAR1 (Adenosine deaminase Acting on RNA) or ADAR2 (Adenosine deaminase Acting on RNA), but not always limited thereto. In a specific embodiment of the present invention, the ADAR protein is ADAR2, and the deaminase domain (ADAR2 276-702) involved in A-to-I RNA editing and the deaminase domain of ADAR2 mutated to enable C-to-U RNA editing (V16S/E488Q/V351G/S486A/T375A/S370C/P462A/N597I/L332I/I398V/K350I/M383L/D619G/S582T/V440I/S495N/K418E/S661T) were used.

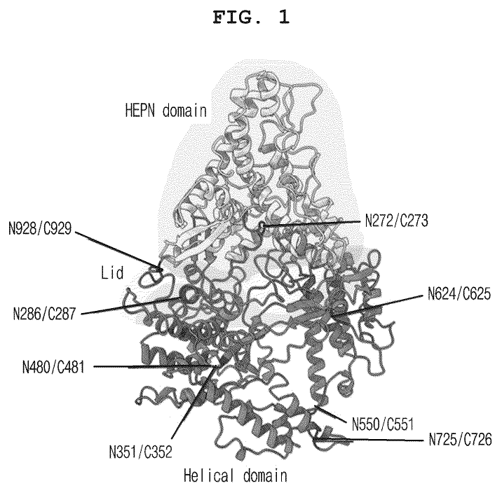

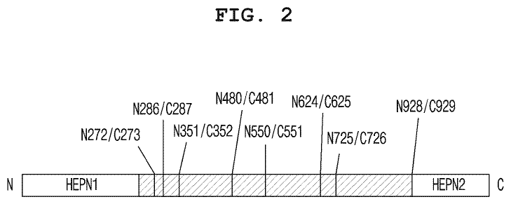

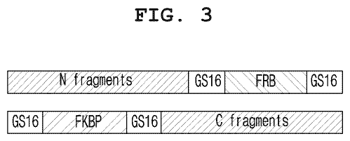

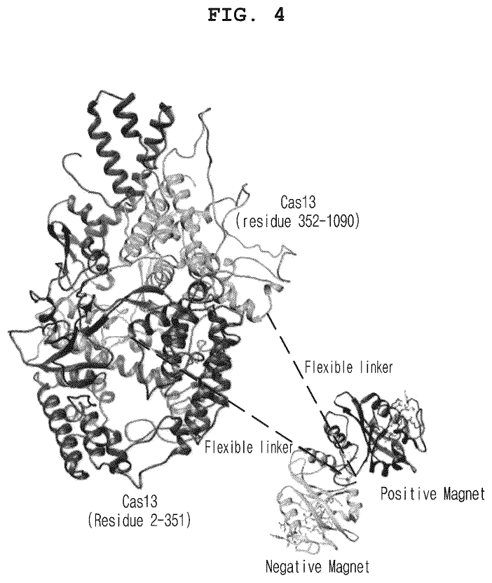

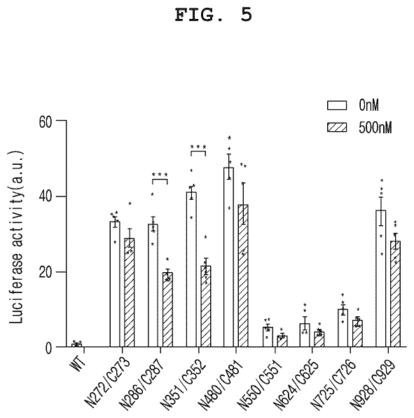

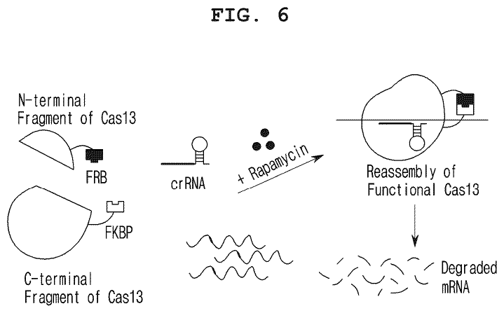

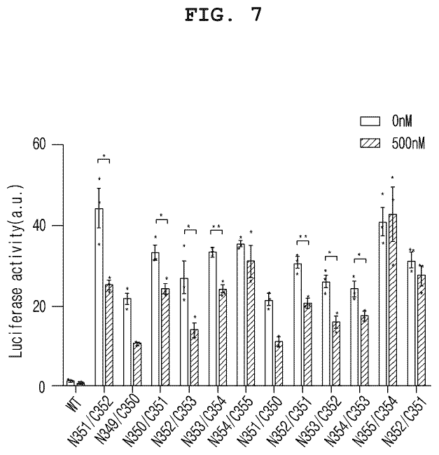

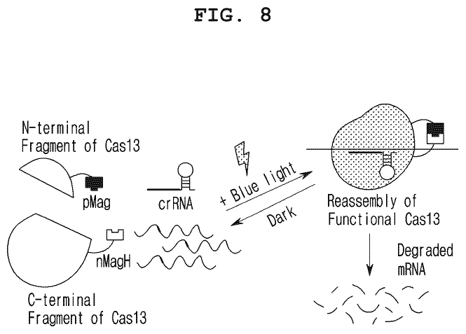

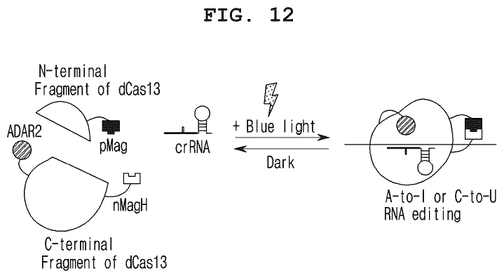

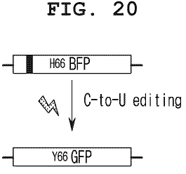

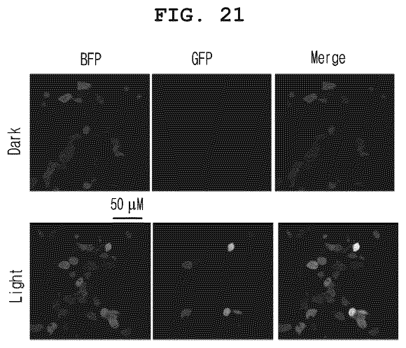

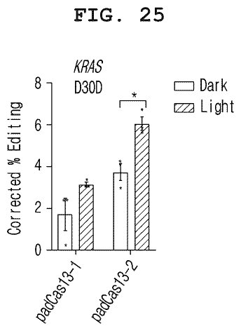

In a specific embodiment of the present invention, a fragment of Cas13b was designed, which is expected to exhibit Cas13b activity because two HEPN domains with RNase activity can be conserved and recombined (FIGS. 1 and 2). A flexible controllable construct was designed by connecting each regulatory protein with a linker (GS16) not to affect Cas13b protein function using the FKBP (FK506-binding protein)-FRB (FKBP-rapamycin-binding) system known to be dimerized by rapamycin (see FIG. 3). The other flexible controllable construct was designed by connecting each regulatory protein with a linker (GS16) not to affect Cas13b protein function using the nMag-pMag system known to be dimerized by blue light (see FIG. 4). By confirming that the luciferase activity was decreased by rapamycin treatment, it was confirmed that the activity of Cas13b protein can be regulated by rapamycin (see FIG. 5), and a chemo-inducible RNA regulation module was developed using this (see FIG. 6). In addition, it was that confirmed Cas13b can be activated by irradiating blue light and that the expression of the target RNA can be reduced by the crRNA that can be hybridized with the Cas13b activated by blue light and the target RNA (FIGS. 8 to 11). As a result of removing the RNase activity and linking ADAR2, an RNA editing regulatory protein, to the C-terminus of Cas13b, it was confirmed that the A-to-I RNA mutation can be edited by blue light in the Firefly reporter, which does not produce a normal protein by the mutation (FIGS. 12 to 20). By designing a fluorescent protein-based indicator, it was also confirmed that C-to-U base editing was possible by blue light (FIGS. 21 to 24). [symbol] , In addition, it was confirmed that the light-induced padCas13 editor can edit endogenous transcripts (FIGS. 25 and 26).

Therefore, the method of the present invention can be effectively used in the treatment of a disease by modulating RNA expression or editing RNA mutation associated with the disease.

Hereinafter, the present invention will be described in detail by the following examples and experimental examples.

However, the following examples and experimental examples are only for illustrating the present invention, and the contents of the present invention are not limited thereto.

### Example 1: Prediction of Cas13b Protein Fragment Candidate Capable of Rebinding Through Reconstruction of PspCas13b Protein

In order to control the activity of Cas13b while targeting the endogenous RNA of animal cells, the PspCas13b (Prevotella sp. P5-125 Cas13b) system known to be strongly expressed in animal cells was used. However, since the structure of PspCas13b was not identified, the structure of PspCas13b was reconstructed by UCSF Chimera tool using the structure of PbuCas13b, which has already been identified (see FIG. 1). By conserving and recombination of two HEPN domains having RNase activity, a fragment of Cas13b expected to exhibit Cas13b activity was designed.

As a result, as shown in FIGS. 1 and 2, 8 fragment candidates of PspCas13b protein were predicted.

### Example 2: Construct Design that can Chemically Modulate PspCas13b Protein Activity

A construct was designed to chemically control the activity of PspCas13b protein based on the recombination-capable fragment of Cas13b protein predicted in Example 1 above.

Specifically, as shown in FIG. 3, a construct was designed using the FKBP (FK506-binding protein)-FRB FRB (FKBP-rapamycin-binding) system known to be dimerized by rapamycin. The cDNA encoding the Cas13b fragment of Example 1 fused with the nuclear export signal of HIV was amplified in pC0046-EF1a-PspCas13b-NES-HIV (Addgene plasmid 103862). Each regulatory protein was connected with a linker (GS16) so as not to affect the Cas13b protein function to enable flexible modulation. The DNA sequence encoding the FKBP-FRB domain was synthesized by Twist Bioscience. All Cas13 fragments were synthesized using Gibson assembly. PCR fragments for Gibson Assembly were amplified using Phusion Hot-Start II DNA Polymerase (Thermofisher). A chemoinducible Cas13 construct based on the GFKBP-FRB domain and the Cas13 fragment (N-terminus and C-terminus) was cloned into the XhoI/XmaI and BsrGI/NotI sites of the EGFP-N1 plasmid (Clonetech), respectively.

### Example 3: Construct Design That can Optogenetically Modulate PspCas13b Protein Activity

A construct was designed to optogenetically control the activity of PspCas13b protein based on the recombination-capable fragment of Cas13b protein predicted in Example 1 above.

Specifically, as shown in FIG. 4, a construct was designed using the nMag-pMag system known to be dimerized by blue light. Since FKBP and FRB are 107 amino acids and 93 amino acids in length, a magnet system (150 amino acids) of similar size was used to avoid steric hindrance. Two combinations of pMag : nMag and pMag:nMagH (nMag with M135I and M165I mutations) were used. To construct padCas13, the inactivated HEPN RNase domain (including H133A and H1058A mutations) was amplified in pC0049-EF1a-dPspCas13b-NES-HIV (Addgene 103865). Each regulatory protein was connected with a linker (GS16) so as not to affect the Cas13b protein function to enable flexible modulation. The DNA sequences encoding the pMag:nMag and pMag:nMagH domains were synthesized by Twist Bioscience. All Cas13 fragments were synthesized using Gibson assembly. PCR fragments for Gibson Assembly were amplified using Phusion Hot-Start II DNA Polymerase (Thermofisher). A photoinducible Cas13 construct based on the pMag:nMag or pMag:nMagH domain and the Cas13 fragment (N-terminus and C-terminus) was cloned into the XhoI/XmaI and BsrGI/NotI sites of the EGFP-N1 plasmid (Clonetech), respectively.

### Example 4: Construction of crRNA

CrRNAs targeting the Fluc reporter, GFP indicator, KRAS, PPIB, STAT3 and A-to-I or C-to-U RNA editing site were generated by annealed oligo cloning using the pC0043-PspCas13b crRNA backbone (Bbsl region of Addgene plasmid 103854). All crRNA plasmids were cloned using T4 DNA ligase (Elpis Biotech). A multiplexed crRNA was constructed using Gibson assembly of three fragment ligation.

### Experimental Example 1: Confirming Whether Cas13b Protein is Activated by Rapamycin

Luciferase reporter assay was performed to confirm whether the Cas13b protein was activated by rapamycin when the construct designed in Example 2 was used.

Specifically, to evaluate the changes in protein levels, HEK293T cells (ATCC) were co-transfected with 10 ng of a double luciferase reporter plasmid (Promega), 75 ng of each inducible Cas13 fragment expressing the plasmid, and 100 ng of a crRNA expression plasmid. 16 hours prior to transfection, the cells were placed in a 96-well plate (Corning) and allowed to grow until reaching 80% confluency. On the next day, 10 μL of Opti-MEM and 10 μL of Opti-MEM containing 0.6 μL of lipofectamine LTX and 0.26 μL of PLUS reagent and all transfected DNA plasmids were mixed, and a total of 20 μL of Opti-MEM I reducing serum medium (ThermoFisher) was added to each well. All solutions were incubated for 5 minutes before slowly added to the cells. For 24 hours prior to analysis, 500 nM of rapamycin was treated and 48 hours later, luciferase readouts of Firefly luciferase (experimental group) and Renilla luciferase (control group) were sequentially measured with a Tecan plate reader using the Dual-Glo Luciferase Assay System (Promega) according to the manufacturer's instructions. The experiment was performed in triplicate. The decrease in the target RNA, the Firefly transcript, was confirmed by comparing the expression value at the protein level of the control group Renilla.

As a result, as shown in FIG. 5, it was confirmed that N550/C551, N624/C625 and N725/C726 among the fragments of Cas13b protein could be reunited in cells without rapamycin stimulation. In addition, it was confirmed that the luciferase expression was moderately reduced in the absence of rapamycin, and the background activity was not due to the residual nuclease activity of the individually cleaved Cas13 fragment. Based on these results, a chemo-inducible RNA regulation module capable of recombination of Cas13 fragments by rapamycin treatment was developed (FIG. 6). In subsequent experiments, in particular, N351/C352 fragments, which were significantly different before and after induction with rapamycin, were used.

A second screening of Cas13 fragment sites was performed near the residues 351 and 352 to select more efficient fragments, and N351/C352, N351/C350 and N353/C352 of paCas13 were named paCas13-1, paCas13-2 and paCas13-, respectively (FIG. 7).

### Experimental Example 2: Confirming Whether Cas13b Protein is Activated by Blue Light

### <2-1> Characterization of Photoactivatable Cas13 (paCas 13)

Luciferase reporter assay was performed to confirm whether the Cas13b protein was activated by light stimulation when the construct designed in Example 3 was used.

To test the light-inducible RNA targeting of paCas13, the target RNA knockdown activity of paCas13 fragments was evaluated using a dual luciferase system under the blue light stimulation for 24 hours with an LED plate emitting 488 nm of blue light (FIG. 8). The luciferase reporter assay was performed as described in Example 4.

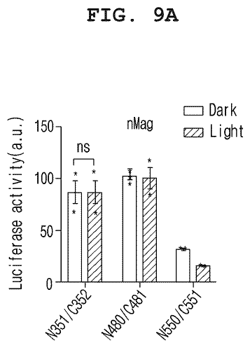

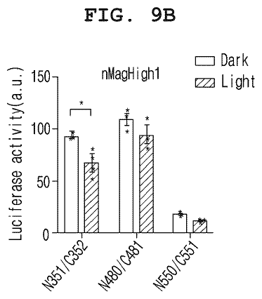

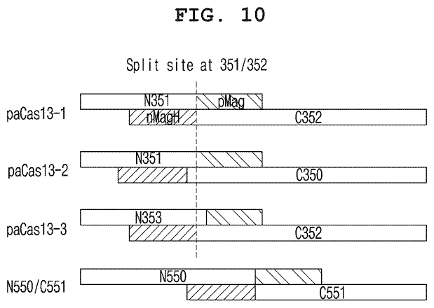

As a result, as shown in FIG. 9, each paCas13 construct showed significant luciferase knockdown upon blue light induction, identical to the rapamycin-inducible construct. Efficient target mRNA cleavage was seen in pMag in combination with nMagH (nMagHigh1) but not nMag. Therefore, in all the subsequent experiments, a system using pMag:nMagH was used.

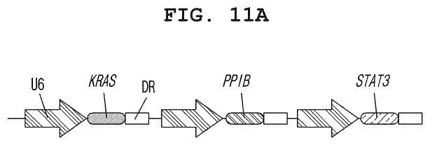

In addition, a light-inducible candidate was selected based on the screening results of Example 4 (FIG. 10), and the experiments to confirm the function of paCas13 on RNA interference by targeting endogenous transcripts, KRAS, PPIB and STAT3 previously targeted by the Cas13 system were performed.

Particularly, to create a system that can achieve efficient and easy multiplex RNA modulation, a single crRNA expression vector with multiple crRNAs was used.

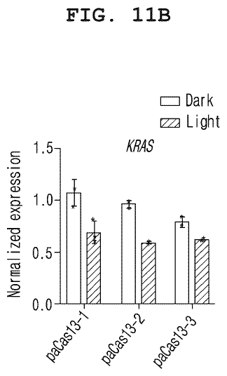

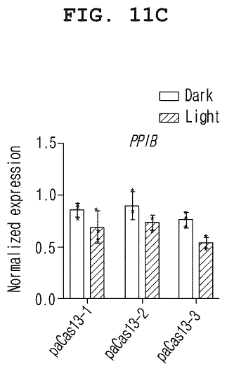

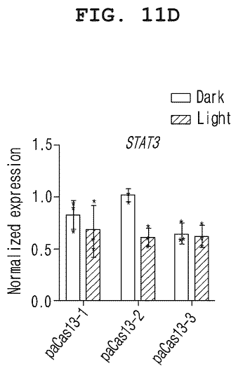

As a result, as shown in FIG. 11, significant knockdown of the target transcript by paCas13 under the light stimulation was observed.

These results indicate that the paCas13 system can perturb endogenous transcripts through light stimulation.

### <2-2> Optogenetic RNA Regulation by paCas13

To further demonstrate the utility of paCas13, a regulatory domain was fused to the padCas13 fragment to construct a photoactivation and reversible RNA editing system (hereinafter referred to as padCas13) (FIG. 12).

Programmable RNA editing systems using catalytically inactive Cas13 (dCas13) to deliver the catalytic domain of ADAR2 (adenosine deaminase acting on RNA type2) to specific transcripts of mammalian cells are known from many studies. ADAR2 hydrolytically deaminates adenosine in double strand RNA to make inosine, and inosine pairs with cytosine and exhibits similar properties to guanosine. In the present invention, the padCas13 system was constructed using the ADAR2 system.

### <2-2-1> Light-Inducible A-to-I RNA Editing by paCas13

To confirm the light-inducible RNA editing of the present invention, A-to-I (adenine to inosine) editing was performed, and then luciferase reporter assay was performed.

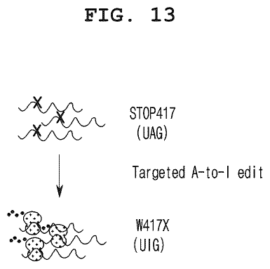

Specifically, the catalytic deaminase domain of ADAR2 for A-to-I editing (ADAR2DD) was amplified in pC0050-CMV-dPspCas13b-longlinker-ADAR2DD (wt) (Addgene plasmid 103866). A padCas13 editor was designed by fusing ADAR2DD to the C terminus of the Cas13 fragment. To verify the activity of the padCas13 editor, an RNA editing reporter for Fluc gene was designed by introducing a nonsense mutation [W417X (UGG→UAG)]. This mutation can be modified to a wild-type codon via A-to-I editing, which can be detected by restoration of the firefly luciferase signal (FIG. 13). The luciferase reporter assay was performed as described in Example 4.

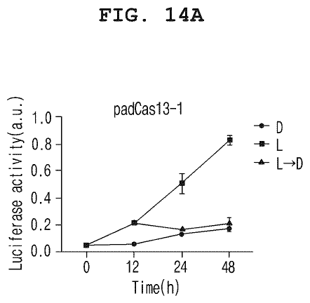

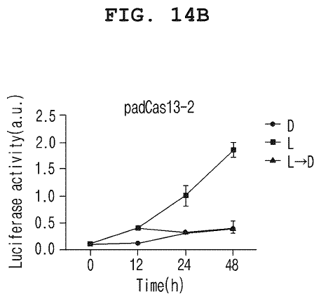

The efficiency of A-to-I editing by light stimulation over time was observed. As a result, as shown in FIG. 14, when blue light was turned on, as the duration of light stimulation was increased, the luciferase activity by restoration was increased. When the light was turned off after stimulation for 6 hours, it was confirmed that the increased luciferase activity by restoration returned to the baseline within 24 hours.

To further verify the restoration of the luciferase activity, reverse transcription (RT)-polymerase chain reaction (PCR) was performed, followed by Sanger sequencing.

Specifically, to evaluate the changes in RNA levels after transfection of the paCas13 system, total RNA was isolated from HEK293T cells and RT-qPCR was used to quantify the changes in RNA levels. Total RNA isolation was performed using PureLink RNA mini kit (Invitrogen). After the RNA isolation, RNA was reverse transcribed into cDNA using gDNA eraser (Takara) and PrimeScript RT Reagent Kit. All qPCR reactions were performed in a volume of 20 μL using SYBR green master mix (Toyobo) and amplified in CFXmaestro 96 (Bio-rad). A qPCR primer was designed to span the crRNA target site. The expression levels were calculated using the cycle threshold (Ct) values of GAPDH and the Ct values of the gene of interest. The relative expression level of one gene was determined as 2^{ΔCt} (ΔCt=Ct (gene of interest)−Ct (GAPDH)). The relative expression level of the target gene was obtained by normalizing the expression level of the target gene in cells treated with on-target crRNA to the expression level of the target gene in cells treated with non-targeting (NT) crRNA.

Sanger sequencing was performed by PCR amplification of the cDNA product with Phusion DNA polymerase (Thermofisher) using target-specific primers, and then by Cosmogintech. Editing efficiency was calculated by analyzing the height of each Sanger peak at the target site using Indigo software (https://www.gear-genomics.com/indigo/). The Sanger sequencing standard curve was generated from the sequencing results of several DNA samples deliberately mixed. A standard curve was used to correct for editing efficiencies that could be altered by PCR bias and sequencing bias.

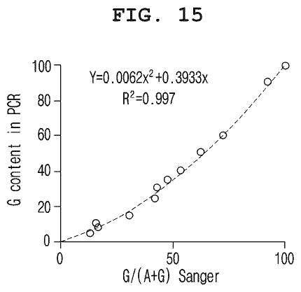

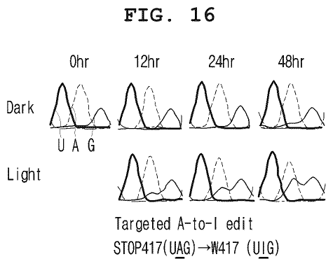

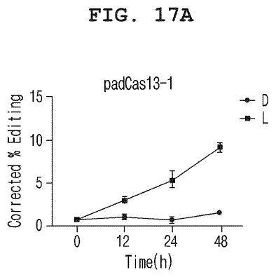

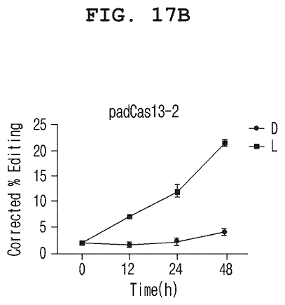

The levels of RNA editing with or without light stimulation and over time were measured for up to 48 hours. As a result, as shown in FIG. 15, it was confirmed that A-to-I RNA editing was performed over time in the presence of light stimulation. In addition, it was confirmed that the RNA editing level of the target site by the padCas13 editor was steadily increased over time (FIGS. 16 and 17). Since the cells transfected with only crRNA also exhibited a moderate A-to-I editing rate, the background activity observed in the dark state was regarded as the RNA editing background unrelated to padCas13 (FIG. 18).

The above results suggest that the padCas13 editor of the present invention is capable of reversible RNA editing in a light-dependent manner.

To evaluate the efficiency and specificity of the padCas13 system of the present invention, transcriptome-wide RNA-sequencing (RNA-seq) was performed on the cells transfected with the padCas13 editor targeting Fluc W417X mutation.

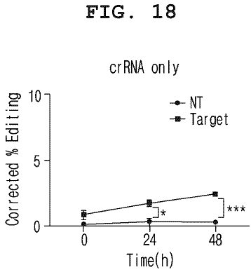

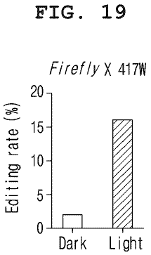

As a result, as shown in FIG. 19, it was confirmed that the target editing rate was 16% under the light stimulation for 24 hours.

The padCas13 editor was able to induce the target A-to-I editing, but many “A” sites around the target site were accessible via the fused ADAR2 of the padCas13 editor. These sites also bound crRNA to form a RNA duplex structure, the natural editing target structure of the endogenous ADAR2 enzyme. Therefore, RNA sequencing was performed to also analyze adenosines surrounding the target mutation site (Fluc W417X).

Specifically, HEK293T cells were plated in 24-well plates (SPL) and co-transfected with 250 ng of each paCas13 fragment expressing the plasmid and 300 ng of crRNA. After the light stimulation, total RNA was extracted using a PureLink RNA mini kit (Invitrogen), and DNase (Invitrogen) treatment was performed in the column for 15 minutes. A strand mRNA library was prepared using a TruSeq stranded mRNA library kit (Illumina) and sequenced at Illumina NextSeq550 with 76 nt paired-end reading. About 130 million total reads per condition were demultiplexed. Sequencing raw data were processed using PIGx RNA-seq (https://github.com/BIMSBbioinfo/pigx rnaseq).

As a result, as shown in FIG. 20, no additional A-to-I editing was observed at adenosine sites other than the target.

These results indicate that the padCas13 editor is capable of light-inducible and highly specific A-to-I RNA editing.

### <2-2-2> Light-Inducible C-to-U RNA Editing by paCas13

A light-inducible C-to-U RNA editing system was constructed by fusion of the padCas13 fragment to ADAR2DD capable of cytidine deamination. To verify the activity of the padCas13 editor for C-to-U nucleotide editing, a fluorescent protein-based indicator was designed (FIG. 21), and fluorescence imaging and flow cytometry were performed.

Particularly, for C-to-U RNA editing, evolved ADAR2 DD mutations (including E488Q/V351G/S486A/T375A/S370C/P462A/N597I/L332I/I398V/K350I/M383L/D619G/S452T/S48) were amplified in pc0079 RESCUE-S (Addgene plasmid 130662). To develop this system, a mutation (Y66H green to blue: Y66H (UAC→CAC)) was introduced into GFP of the EGFP-N1 plasmid by Gibson Assembly. In addition, a crRNA targeting the target CAC codon causing C-to-U conversion was designed. Under the light stimulation, the padCas13 editor can perform C-to-U conversion of Y66H mutation and restore GFP fluorescence. To enhance the effect of C-to-U RNA editing, two vectors were cloned by Gibson assembly following expansion PCR cloning. The first vector expresses the N-terminus of crRNA and the pMag-conjugated dCas13 fragment linked to a GFP indicator via a T2A self-cleaving peptide. Each sequence is controlled by hU6 promoter and EFS (EF1a short) promoter, respectively. The second vector expresses the C terminus of the dCas13 fragment fused with nMagH and ADAR2DD under the control of the EFS promoter.

Live-cell imaging was performed with a Nikon A1R confocal microscope mounted on a Nikon Eclipse Ti body equipped with a Nikon CFI PlanApochromat VC 60X/1.4 numerical aperture (NA) and a digital-zooming Nikon imaging software (NIS-elements AR 64-bit version 3.21). A Chamlide TC system placed on a microscope stage was used to maintain environmental conditions of 37° C. and 10% CO₂ (Live Cell Instruments). A custom 96-well 488 nm LED array (Live Cell Instruments) was used to irradiate uniform blue light in all wells.

To perform flow cytometry, HEK293T cells were seeded in a 6-well plate (SPL) at the density of 3×10⁵ cells/well using a GFP indicator. After 24 hours, the GFP indicator and padCas13 editor plasmids were co-transfected using Lipofectamine LTX (Invitrogen) according to the manufacturer's manual. The percentage of GFP+ in cells indicates editing efficiency. The transfected cells were analyzed by BD FACSLSRFortessa 24 hours after the light stimulation. Only live single cells were used for fluorescence analysis. The results of flow cytometry were analyzed with FlowJo X (v. 10.0.7).

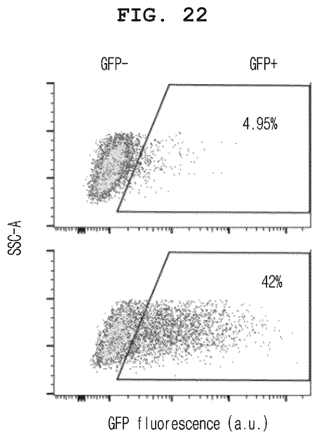

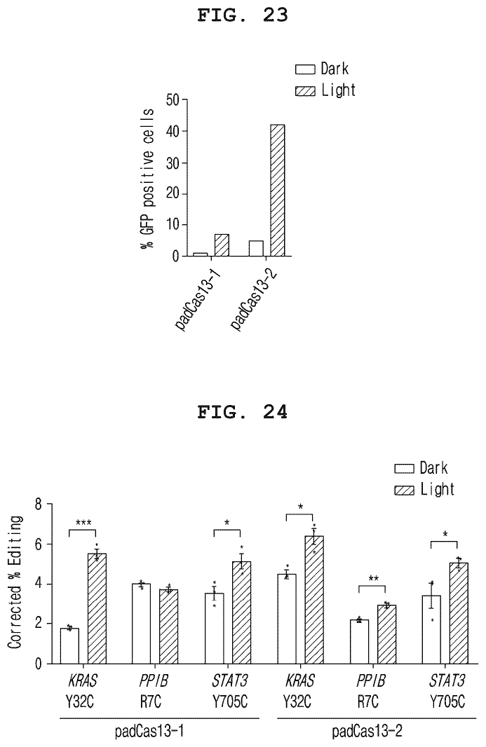

The padCas13 editor and the target crRNA or non-target crRNA (NT, negative control) were transfected with the GFP indicator and the light inducibility upon blue light stimulation was tested for 24 hours. As a result, as shown in FIG. 22, it was confirmed that the padCas13 editor induced C-to-U RNA editing to generate BFP/GFP double positive cells under the light stimulation. It was also confirmed that the GFP restoration caused by C-to-U editing was increased from 4.95% to 42% by blue light illumination compared to the dark condition (FIGS. 23 and 24).

Through the above results, it was confirmed that the padCas13 system of the present invention has a light-dependent RNA editing activity suitable for both A-to-I and C-to-U nucleotide editing.

### <2-2-3> Light-Inducible Endogenous Transcript Editing by padCas13

Whether the light-inducible padCas13 editor of the present invention can be applied to edit endogenous transcripts was evaluated.

As a result, as shown in FIGS. 25 and 26, it was confirmed that the padCas13 editor of the present invention can mediate A-to-I and C-U editing of all the subjects tested in a light-induced manner.

These results indicate a high possibility of using padCas13 to ameliorate a disease. In particular, the use of padCas13 has a h high potential to improve the disease state that requires temporal regulation of disease-related RNA molecules.

What is claimed is:

1. A method for regulating the activity of Cas13 protein, comprising a step of treating a first fragment containing the N-terminal segment of Cas13 protein and a first regulatory protein; and a second fragment containing a second regulatory protein and the C-terminal segment of Cas13 protein with a compound or light irradiation.

2. The method for regulating the activity of Cas13 protein according to claim 1, wherein the compound treatment or light irradiation induces dimerization of the first regulatory protein and the second regulatory protein.

3. The method for regulating the activity of Cas13 protein according to claim 1, wherein a linker is inserted between the N-terminal segment of Cas13 protein and the first regulatory protein, and between the second regulatory protein and the C-terminal segment of Cas13 protein.

4. The method for regulating the activity of Cas13 protein according to claim 3, wherein the linker includes glycine (G) and serine (S).

5. The method for regulating the activity of Cas13 protein according to claim 1, wherein the Cas13 protein is at least one selected from the group consisting of Cas13a, Cas13b, Cas13c and Cas13d.

6. The method for regulating the activity of Cas13 protein according to claim 5, wherein the Cas13 protein is PspCas13b protein.

7. The method for regulating the activity of Cas13 protein according to claim 1, wherein the N-terminal segment of Cas13 protein is a segment from the N-terminus to the 272^{th}, 286^{th}, 351^{th}, 480^{th}, 550^{th}, 624^{th}, 725^{th} or 928^{th} amino acid of Cas13 protein.

8. The method for regulating the activity of Cas13 protein according to claim 1, wherein the C-terminal segment of Cas13 protein is a segment from the C-terminus to the 861^{th}, 847^{th}, 782^{th}, 653^{th}, 583^{th}, 509^{th}, 408^{th} or 205^{th} amino acid of Cas13 protein

9. The method for regulating the activity of Cas13 protein according to claim 1, wherein the compound is rapamycin.

10. The method for regulating the activity of Cas13 protein according to claim 1, wherein the light is blue light.

11. The method for regulating the activity of Cas13 protein according to claim 1, wherein the combination of the first regulatory protein and the second regulatory protein is FKBP-FRB, nMag-pMag or nMagH-pMag.

12. A method for modulating the expression of a target RNA comprising the following steps:
1) preparing a vector comprising a construct containing a sequence encoding the N-terminal segment of Cas13 protein and a sequence encoding a first regulatory protein; and a vector comprising a construct containing a second fragment comprising a sequence encoding a second regulatory protein and a sequence encoding the C-terminal segment of Cas13 protein;
2) injecting the vector above and crRNA (CRISPR RNA) capable of hybridizing to the target RNA sequence into mammalian cells; and
3) treating the cells with a compound or irradiating with light.

13. The method for modulating the expression of a target RNA according to claim 12, wherein the crRNA includes a guide sequence and a direct repeat sequence capable of hybridizing to the target RNA sequence.

14. The method for modulating the expression of a target RNA according to claim 12, wherein the vector is a virus or a plasmid.

15. The method for modulating the expression of a target RNA according to claim 14, wherein the virus is adeno-associated virus (AAV), adenovirus, retrovirus, lentivirus, modified vaccinia virus ankara (MVA), herpes simplex virus or baculovirus.

16. A method for editing the target RNA sequence comprising the following steps:
1) preparing a vector comprising a construct containing a sequence encoding the N-terminal segment of Cas13 protein and a sequence encoding a first regulatory protein; and a vector comprising a construct containing a second fragment comprising a sequence encoding a second regulatory protein, a sequence encoding the C-terminal segment of Cas13 protein and a sequence encoding a RNA editing protein;
2) injecting the vector above and crRNA (CRISPR RNA) capable of hybridizing to the target RNA sequence into mammalian cells; and
3) treating the cells with a compound or irradiating with light.

17. The method for editing the target RNA sequence according to claim 16, wherein the Cas13 protein is a protein from which RNase activity is removed.

18. The method for editing the target RNA sequence according to claim 16, wherein the RNA editing protein is ADAR (adenosine deaminase acting on RNA).
